(19) United States
(12) Patent Application Publication
(10) Pub. No.: US 2005/0260287 A1
(43) Pub. Date: Nov. 24, 2005

(54) Natural hormone replacement therapy

(76) Inventor: Giovina DiMatteo-Leggio, Aventura, FL (US)

Correspondence Address: GIOVINA INTERNATIONAL INC. OF AMERICA, 19495 BISCAYNE BOULEVARD # 807, AVENTURA, FL, 33180, US

(21) Appl. No.: 10/628,348

(22) Filed: May 21, 2004

(51) Int. Cl. A61K 35/78

(52) U.S. Cl. 424/725; 424/773; 424/765; 424/729; 424/745; 424/766; 424/752; 424/764

(57) ABSTRACT

The supplements are a combination of natural herbs, vitamins and minerals that help alleviate symptoms and risks related to menopause. They are packaged in bottles of 180 tablets. The supplements are in the form of tablets of which five tablets daily are taken orally.

Published scientific literature confirm that certain plant compounds used in our formulas produce progestational and estrogenic effects by putting mild plant estrogens or plant hormones back into the system, which reportedly help to offset menopause's estrogen deficit.

Our recommended diets contain estrogenic plant components that also may help stem hot flashes and other symptoms of menopause, by adding to estrogen levels in the body.

Some plant factors obtained through diet might also reduce the risk for cancer: by binding to and blocking estrogen receptors, thus diminishing the effects of incoming natural estrogen that other—wise might lead to tumor growth. (Tamoxifen, an estrogenlike compound and a treatment for breast cancer, is thought to work in this manner.)

1. I claim that our Hormone Replacement Therapy significantly alleviates peri-menopausal and menopausal symptoms and risks associated with menopause and, guides in implementing a healthy lifestyle.
